(19) United States
(12) Patent Application Publication
(10) Pub. No.: US 2025/0107945 A1
(43) Pub. Date: Apr. 3, 2025

(54) UTILIZATION OF MULTIVARIABLE REAL-TIME & CALIBRATION DATA INFERENCING TO IMPROVE THE PREDICTION OF A DISPOSABLE DIAPERS SATURATION LEVEL

(71) Applicant: Matthew Napier, Cedar City, UT (US)

(72) Inventor: Matthew Napier, Cedar City, UT (US)

(21) Appl. No.: 18/899,933

(22) Filed: Sep. 27, 2024

Related U.S. Application Data
(60) Provisional application No. 63/586,168, filed on Sep. 28, 2023.
(60) Provisional application No. 63/586,177, filed on Sep. 28, 2023.
(60) Provisional application No. 63/586,179, filed on Sep. 28, 2023.

(51) Int. Cl. A61F 13/42; G01N 27/04; G01N 27/22; G16H 40/67

(52) U.S. Cl. CPC A61F 13/42; G01N 27/048; G01N 27/223; G16H 40/67; A61F 2013/424

(57) ABSTRACT

A method for determining a diaper saturation level. The method includes receiving a message from a front-end sensor and processing the message. The method also includes checking the message for flags and determining if the diaper has been disconnected from the sensor. The method further includes resetting the saturation level to zero if the diaper has been disconnected and estimating the saturation level if the diaper was not disconnected from the sensor.

### CROSS-REFERENCE TO RELATED APPLICATIONS

This application claims the benefit of and priority to U.S. Provisional Patent Application Ser. No. 63/586,179 filed on Sep. 28, 2023, which application is incorporated herein by reference in its entirety.

This application is related to co-pending U.S. Patent application Ser. No. ______, filed on Sep. 27, 2024 (Attorney Docket No. 10536-10), and entitled, “THE UTILIZATION OF AN ADAPTABLE LOW POWER SENSOR TO ENABLE SIMULTANEOUS IN-WET EVENT DEVICE 100 HIGH DYNAMIC RANGE MEASUREMENT OF RESISTANCE AND CAPACITANCE TO ESTIMATE AND INTELLIGENTLY TRANSMIT THE SATURATION OF A SENSOR ENHANCED DISPOSABLE DIAPER”, which application is incorporated herein by reference in its entirety (hereinafter “first related application”).

Co-pending application Ser. No. ______, (Attorney Docket No. 10536-10) claims the benefit of and priority to U.S. Provisional Patent Application Ser. No. 63/586,168 filed on Sep. 28, 2023 which application is incorporated herein by reference in its entirety.

This application is related to co-pending U.S. Patent application Ser. No. ______, filed on Sep. 27, 2024 (Attorney Docket No. 10536-11), and entitled, “SIMULTANEOUS RESISTANCE AND CAPACITANCE MEASUREMENT AND INFERENCING ALGORITHM ENABLING REAL TIME AND IMPROVED INCONTINENCE WET SENSING AND DIAPER SATURATION ESTIMATION”, which application is incorporated herein by reference in its entirety (hereinafter “first related application”).

Co-pending application Ser. No. ______, (Attorney Docket No. 10536-11) claims the benefit of and priority to U.S. Provisional Patent Application Ser. No. 63/586,177 filed on Sep. 28, 2023 which application is incorporated herein by reference in its entirety.

### BACKGROUND OF THE INVENTION

The number of incontinent individuals in the world is rising significantly encompassing the aging population, babies/toddlers, and those who are handicapped/disabled. A cost-effective way to estimate the saturation of a disposable diaper will significantly enhance the quality of life for those people with significant incontinence. Quick changes of a diaper, after an incontinent event, will reduce the risk of skin irritation, UTI's and other diaper related ailments. What is needed is a simple and cost-effective sensor placed directly into the diaper which gives the user or a caregiver information about diaper wetness/saturation. The removable sensor attaches via snaps (or other attachment method) which allows quick and easy diaper changes. Our advanced sensing methods enable this cost-effective solution at a price point lower than competitors.

Sensing the saturation of a diaper with one sensor modality makes it very difficult to repeatedly predict the saturation of a diaper. This multi sensing modality software, when married to a multi sensing front-end sensor, enables multiple sensing modalities to be generated, transmitted and received via IoT message and inferred into a diaper saturation estimate.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a method for determining a diaper saturation level. The method includes receiving a message from a front-end sensor and processing the message. The method also includes checking the message for flags and determining if the diaper has been disconnected from the sensor. The method further includes resetting the saturation level to zero if the diaper has been disconnected and estimating the saturation level if the diaper was not disconnected from the sensor.

Another example embodiment includes a method for determining a diaper saturation level. The method includes receiving a message from a front-end sensor and processing the message. The method also includes checking the message for flags and determining if the diaper has been disconnected from the sensor. The method further includes resetting the saturation level to zero if the diaper has been disconnected and estimating the saturation level if the diaper was not disconnected from the sensor. The method additionally includes determining whether a user should be notified about the saturation level of the diaper and notifying a user of the saturation level if the determination of whether a user should be notified is positive.

Another example embodiment includes a system for determining a saturation level in a diaper. The system includes a front-end sensor configured to be placed within a diaper. The front-end sensor is configured to allow a simultaneous estimation of the resistance within the diaper and the capacitance within the diaper. The system also includes a software application on an external device. The software application receives a message from a front-end sensor and processes the messages. The software application also checks the messages for flags and determines if the diaper has been disconnected from the sensor. The software application further resets the saturation level to zero if the diaper has been disconnected and estimates the saturation level if the diaper was not disconnected from the sensor. The software application additionally determines whether a user should be notified about the saturation level of the diaper and notifies a user of the saturation level if the determination of whether a user should be notified is positive.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of a block diagram of a front-end sensor that can be used to measure both resistance and capacitance of the within a diaper; and

FIG. 2 is a flow chart illustrating a method of determining a diaper saturation level.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

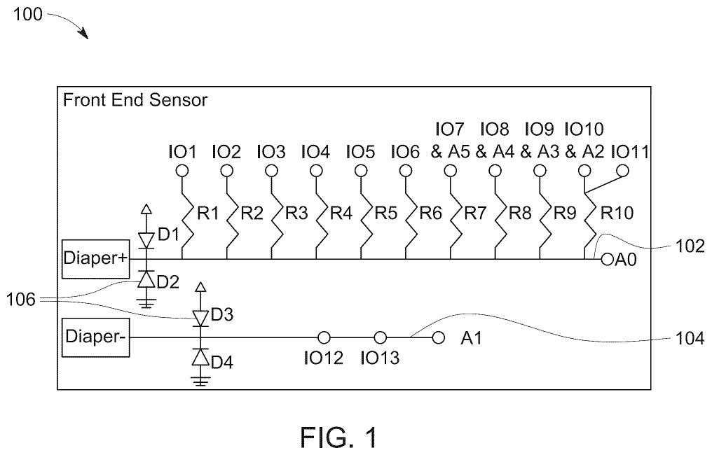

FIG. 1 illustrates an example of a block diagram of a front-end sensor 100 that can be used to measure both resistance and capacitance of the within a diaper. The front-end sensor 100 is placed within the diaper either as an add-on or built into the diaper. In particular, the front-end sensor 100 is placed within the diaper environment. The front-end sensor 100 can be built into the diaper and sold as a single unit or can be sold as a separate device which is placed within a diaper by a user.

FIG. 1 also shows that the front-end sensor 100 can include a first conducting strip 102. The first conducting strip 102 runs the length of the diaper (front to back). The first conducting strip 102 includes a pair of diodes and a series of resistors connected to input/outputs.

FIG. 1 further shows that the front-end sensor 100 can include a second conducting strip 104. The second conducting strip 104 runs the length of the diaper (front to back). The second conducting strip 104 includes a pair of diodes and a series of inputs/outputs.

The combination of the first conducting strip 102 and the second conducting strip 104 allows for the estimation of the resistance within the diaper environment. This allows the wet event device 100 to determine when resistance within the diaper environment has changed. Moisture within the diaper environment, especially sudden changes in moisture, changes the resistance. However, a change in resistance by itself doesn't indicate a wet event because other changes in the environment can cause a change in resistance. For example, if the user is sweating and the sweat runs down the back of the diaper and interacts with the front-end sensor 100, the resistance measurement would be similar to the resistance change due to a wet event. However, this would not require a diaper change. Therefore, changes in resistance as measured by the first conducting strip 102 can indicate a wet event but are not by themselves definitive.

Likewise, the combination of the first conducting strip 102 and the second conducting strip 104 allows for an estimate of the capacitance within the diaper environment. This allows the wet even device 100 to determine when capacitance within the diaper environment has changed. Moisture within the diaper environment, especially sudden changes in moisture, changes the capacitance. However, a change in capacitance by itself doesn't indicate a wet event because other changes in the environment can cause a change in capacitance. For example, in the swat example above, the sweat would create a combination of low resistance and low capacitance whereas a wet event would create low resistance and high capacitance. Further, someone standing in a wet diaper would lead to a different capacitance measurement than that same person sitting in that same diaper. Therefore, changes in capacitance as measured by the second conducting strip 104 can indicate a wet event but are not by themselves definitive.

FIG. 1 additionally shows that the front-end sensor 100 can include electrostatic discharge (ESD) clamp diodes 106 on the first conducting strip 102 and the second conducting strip 104. The ESD clamp diodes 106 protect the first conducting strip 102 and the second conducting strip 104 from the harsh environment of the diaper. In particular, the ESD clamp diodes 106 prevent stray electrical signals (e.g., from the user's body, or from static electricity from rubbing against clothing or the user's body) from creating false signals and damage to the first conducting strip 102 and the second conducting strip 104. In some cases, the Diaper Minus connection is tied directly to sensor ground which removes the need for the ESD clamp diodes 106. Sensor ground is a common reference point for all electronics on the sensor and is not to be confused with earth ground.

The first conducting strip 102 and the second conducting strip 104 can create a multi-component pull up resistance circuit which enables: (1) a high dynamic range diaper resistance measurement estimate, (2) the in-wet event device 100 step response parallel Thevenin equivalent resistance estimation and (3) a high dynamic range capacitance measurement estimate. A high dynamic range pull up resistance circuit enables high dynamic range capacitance measurement estimates in the presence of parallel diaper resistance. High dynamic range measurement of both the resistance and the capacitance allows the inference of the diaper over a wide range of states, from disconnected through fully saturated.

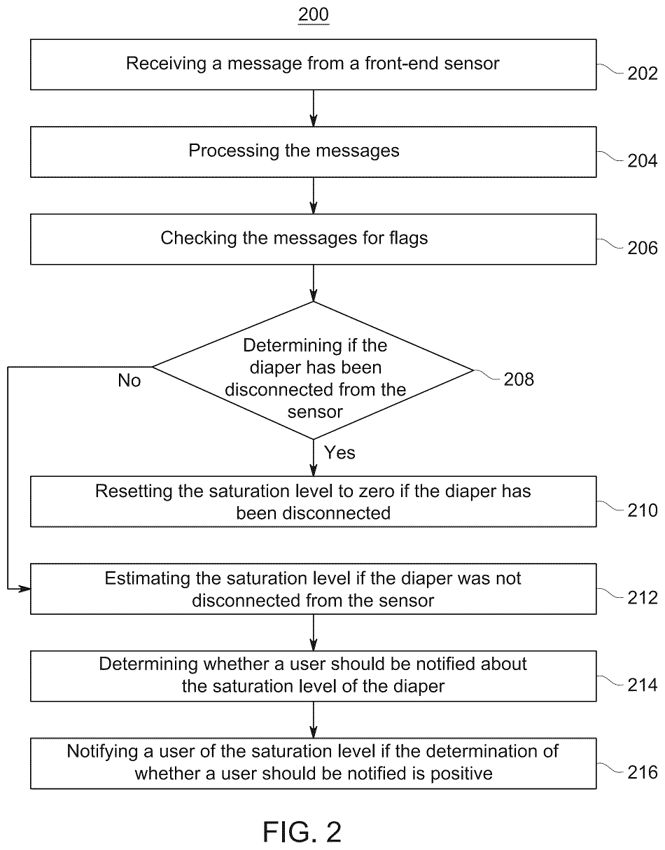

FIG. 2 is a flow chart illustrating a method 200 of determining a diaper saturation level. In at least one implementation, the method can use a sensor, such as the front-end sensor 100 of FIG. 1. Therefore, the method 200 will be described, exemplarily, with reference to the front-end sensor 100 of FIG. 1. Nevertheless, one of skill in the art can appreciate that the method 200 can be used to produce a determination about diaper saturation using a front-end sensor other than the front-end sensor 100 of FIG. 1.

The method 200 allows for wetness/saturation sensing of a disposable undergarment. Detecting the saturation of a diaper is challenging due to the dynamically changing environment that the diaper is exposed to and experiences. This dynamic environment presents itself to the sensor as multiple random noise sources that can be quite large and of variable duration, impacting both the steady state and impulse response of the sensor measurements. Periodically sampling the diaper environment in both a steady state and dynamic manner enables a statistically significant data set of measurements to be collected, enabling processing algorithms to effectively process the saturation level of the diaper. Applying parameterized statistical methods at the front end of the diaper wet sensing chain enables a more reliable and repeatable instantaneous diaper saturation estimate. This requires filtering and suppressing general noise and electrostatic discharge (ESD) events commonly experienced in a disposable diaper environment.

FIG. 2 shows that the method 200 can include receiving 202 a message from a front-end sensor. The message includes data about the diaper resistance, the diaper capacitance, the percent change from the previous resistance and capacitance measurement, the sensor pull up resistance used, and the sensor sample count used to determine the capacitance. This information allows for the saturation level of the diaper to be determined. As messages are received by the back-end cloud processing each diaper resistance and capacitance result is analyzed and saturation estimate determined. This provides a continuous picture of the saturation level of the diaper allowing for a determination of when changes occur.

FIG. 2 also shows that the method 200 can include processing 204 the messages. Messages are processed 204 in the order that they are received. This is important because processing the message depends in part on the prior state of the diaper. I.e., the current state of the diaper is not as accurately estimated unless the prior state is known, so messages need to be processed 204 in order. When a message is received the fields are parsed. Messages are binned based on unique identifier (UID). Messages of the same UID are processed in sequential message order. Out of order messages are delayed processing for a predetermined number of seconds to see if the missing message is received. Messages that start with sequence number “1” represent a wet sensor module reset and are processed immediately regardless of order. Based on the current message resistance, capacitance and message flags down stream processing steps are determined. In addition, messages are used to discover whether a disconnect event has occurred.

FIG. 2 further shows that the method 200 can include checking 206 the messages for flags. Flags indicate a state change that can be used to help determine the current state of the diaper. For example, possible flags can include a diaper change flag, which indicates that the diaper has been changed. If the diaper was changed in the recent past, then the saturation level can be estimated to be zero. Notification to the caregiver is sent confirming diaper change. In addition, the patient's available diaper count is decremented by one. Likewise, possible flags can include a saturation change flag. If the diaper saturation change flag is set it has been determined that the diaper experienced a wet event that changed both the resistance and capacitance from previous measurement enough to warrant a message be generated and sent. If the wet event was small then the change may not be enough to warrant message notification. The diaper saturation flag indicates the saturation state which means that the resistance and capacitance have changed enough to alert the back end. This flag forces a message to be sent from the wet module and saturation estimation processing is initiated. Other possible flags could include status flags such as a sensor attachment issue flag.

FIG. 2 additionally shows that the method 200 can include determining 208 if the diaper has been disconnected from the sensor. If the diaper has been disconnected, that means that the diaper is being changed or was recently changed. If the diaper has not been disconnected then measuring of the diaper saturation level should continue.

FIG. 2 moreover shows that the method 200 can include resetting 210 the saturation level to zero if the diaper has been disconnected. It can be assumed that right after a diaper change and placement of a new sensor, that the diaper is dry, and that the saturation level is zero. This allows for the first measurement of the diaper state to be correlated to a saturation level of zero and calibration of the sensor readings.

FIG. 2 also shows that the method 200 can include estimating 212 the saturation level if the diaper was not disconnected from the sensor. In particular, current diaper resistance and capacitance measurements are compared to values in one or more saturation look up tables. The diaper saturation estimation will be determined by a series of look up tables (LUT) and series of logic decision trees on which LUT to utilize, as described below. There will be multiple LUTs utilized during the saturation estimation processing. The different LUTs will address the differences in measurement associated with two major factors: 1) urine salinity (sodium chloride) which will impact the resistance curve more than the capacitance; and 2) Diaper pulp compression (standing vs sitting/lying) which will impact the capacitance curve more than the resistance. The initial list of LUTs that will be generated is below in Table 1. The LUTs are generated and updated based on data compiled during actual sensor use. The LUTs below are exemplary and do not necessarily represent the data in the LUTs generated by actual data. The first LUT, table #0, contains the lookup values for a dry or disconnected sensor. The remaining LUTs, tables #1 through #10 are LUT tables based on different urine salinity and diaper compression states.

TABLE 1
LUTs that can be selected to estimate saturation levels

| | | | Sodium Chloride | | |
|---|---|---|---|---|---|
| | | | mEq/ | mg/ | tsp/ |
| LUT ID | Salinity | Compression | 32 oz | 32 oz | 32 oz |
| “DisconnectLUT” | N/A | N/A | 0 | 0 | 0 |
| “DryLUT” | N/A | N/A | 0 | 0 | 0 |
| “SweatLUT” | N/A | N/A | 0 | 0 | 0 |
| “LowUnCompLUT” | Low | uncompressed | 0 | 0 | 0 |
| “LowCompLUT” | Low | compressed | 0 | 0 | 0 |
| “NomUnCompLUT” | Nominal | uncompressed | 20 | 460 | 0.092 |
| “NomCompLUT” | Nominal | compressed | 20 | 460 | 0.092 |
| “MedUnCompLUT” | Medium | uncompressed | 135 | 3105 | 0.621 |
| “MedCompLUT” | Medium | compressed | 135 | 3105 | 0.621 |
| “HihUnCompLUT” | High | uncompressed | 270 | 6210 | 1.242 |
| “HihCompLUT” | High | compressed | 270 | 6210 | 1.242 |

Conversions: 1 mg = 0.0002 tsp

Once the LUT is selected, the LUT is used for the life of the diaper. This allows for consistent comparisons between the current diaper state and previous diaper states. Table 2 illustrates an example of a potential saturation LUT.

TABLE 2
example saturation LUT

| | | | Diaper | |
|---|---|---|---|---|
| | | Pull Up | Fluid | Diaper |
| Diaper CAP | Diaper RES | RES | Capacity | Saturation |
| (PicoFarads) | (Ohms) | (Ohms) | (oz) | (%) |
| 1311455 | 4842 | 1000 | 3.2 | 10.00% |
| 1792072 | 3240 | 1000 | 6.4 | 20.00% |
| 1913833 | 2978 | 1000 | 9.6 | 30.00% |
| 2716794 | 1808 | 1000 | 12.8 | 40.00% |
| 3758285 | 1216 | 1000 | 16 | 50.00% |
| 4848099 | 860 | 1000 | 19.2 | 60.00% |
| 5617142 | 723 | 1000 | 22.4 | 70.00% |
| 5648483 | 714 | 1000 | 25.6 | 80.00% |
| 5667814 | 712 | 1000 | 28.8 | 90.00% |
| 5829562 | 700 | 1000 | 32 | 100.00% |

A disconnected sensor has different resistance and capacitance readings of either a saturated diaper or a connected but dry diaper. In both cases saturation is zero and resistance is high (because there is no moisture to conduct any electrical signals) but the capacitance is different. These are shown in Tables 3 and 4.

TABLE 3
example disconnected sensor LUT

| | | | Diaper Fluid | Diaper |
|---|---|---|---|---|
| Diaper CAP | Diaper RES | Pull Up RES | Capacity | Saturation |
| (PicoFarads) | (Ohms) | (Ohms | (OZ) | (%) |
| 85 | 1.00E+06 | 1.00E+06 | 0 | 0.00% |

TABLE 4
example dry diaper LUT

| | | | Diaper Fluid | Diaper |
|---|---|---|---|---|
| Diaper CAP | Diaper RES | Pull Up RES | Capacity | Saturation |
| (PicoFarads) | (Ohms) | (Ohms) | (oz) | (%) |
| 120 | 1.00E+06 | 1.00E+06 | 0 | 0.00% |

The LUTs are generated via controlled tests w/the above listed salinity and compression of the diaper. Key to these LUTs is the measurement of both the resistance and capacitance as the values change differently if the diaper is wet and uncompressed or wet and compressed. I.e., both the wetness and the amount of compression change the resistance and the capacitance making saturation determinations difficult. In addition, the salinity also impacts the baseline values of both resistance and capacitance and any wetness detection system should be able to account for each of these factors to generate a more accurate saturation estimate.

To do this, the proper LUT is selected which allows for a more accurate determination of saturation. If the diaper was dry in the previous message and wet in the current message the back-end processing compares the current (wet) measurement with all LUT rows in the system and finds the closest match. Once a LUT has been selected only this LUT and its compressed counterpart will be used for the life of this diaper. Subsequent messages will be processed against these two LUTs, which significantly improves (decreases) the amount of processing required to calculate the saturation estimate.

The saturation level of the diaper is calculated by comparing the current resistance, capacitance and pull up resistance with values in the LUT and finding the closest matching pair. The measurement is done by a percent change least squares calculation of the current message diaper resistance and capacitance and the LUT values. The least square terms are normalized with the LUT value terms to equally weight the terms. An example of pseudo code for the calculation is shown:

| | |
|---|---|
| 1. | FOR(j=0 j<LUTRows j++) |

| | |
|---|---|
| a. | For each LUT row do “least square” measurement to find the closest point |
| a. | IF(Cur/LUTRow.PullUp == CurPullUp |
| b. | CurLUTRow = readRow(CurLUTFile) |
| c. | %DeltaRes = (CurRes − CurLUTRow.RES)/CurLUTRow.RES |
| d. | %DeltaCap = (CurCap − CurLUTRow.CAP)/CurLUTRow.CAP |
| e. | CurDistance = sqrt( %DeltaRes\hat{ }2+%DeltaCap\hat{ }2+%DeltaPullUp\hat{ }2) |
| f. | IF(CurDistance < CurrentClosestDistance) |

| | |
|---|---|
| i. | Store the current closest point |
| ii. | CurrentClosestDistance = CurDistance |
| iii. | CurrentClosestLUT = LUT_List[i] |
| iv. | CurrentClosestRow = j+1 |

After the initial closest fit search across all LUTs in the system the algorithm sets the LUT space to the LUT that was selected. If the LUT selected was non-compressed it will also utilize the same sodium concentration LUT for the compressed case. All future closest fit searches will be limited to these two LUT tables for the remainder of the current diaper's life. This accomplishes two purposes: 1) smaller search area enabling faster closest fit determination; and 2) better saturation estimation of the diaper based on initial sodium content.

Using the selected LUT the saturation level is determined. If the current saturation value is less than the previous selection by less than a threshold amount the current saturation value is maintained. The threshold amount will generally be determined by testing and can be changed as needed. For example, the threshold amount could be set at 40% and then updated as needed to ensure the proper balance between battery life and notifications to the user. This is required because the diaper is a very dynamic system and after a wet event the diaper material will absorb the urine differently based on a number of factors, but initial wet measurement of the most recent event best estimates the current overall saturation of the diaper.

FIG. 2 further shows that the method 200 can include determining 214 whether a user should be notified about the saturation level of the diaper. Whether to notify a user is a tradeoff because each notification has a power requirement. I.e., every time the user is notified, there is a cost in battery power. In addition, if users are given too many notifications, then they begin to ignore all notifications. Thus, notifications should not be sent for every change in saturation level. The determination 214 of whether a user should be notified is a combination of user set saturation thresholds, minimum notification levels, whether the user has disabled certain notifications (such as a diaper change notification), present saturation change levels, etc.

FIG. 2 additionally shows that the method 200 can include notifying 216 a user of the saturation level if the determination 214 of whether a user should be notified is positive. Once a saturation level is determined, the user (caregiver, wearer, etc.) will be updated via an IOS/Android app or any other software as a service platform (web, 3rd party application) of the diaper's current saturation state. For example, if the diaper returns to dry or is significantly less saturated (i.e., close to dry) the back-end algorithm will reset the saturation level and notify 216 the user of the change and ask for confirmation that the diaper was changed. Likewise, the app will notify 216 the user when diaper saturation exceeds a threshold amount, which can be set by the user.

In addition, the app provides a history panel/screen enabling the user to see the saturation history of the current diaper. In addition, the user can review previous days/weeks usage history. Finally, the app will inference the history data and alert user of anomalies.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for determining a diaper saturation level, the method comprising:
receiving a message from a front-end sensor;
processing the message;
checking the message for flags;
determining if the diaper has been disconnected from the sensor;
resetting the saturation level to zero if the diaper has been disconnected; and
estimating the saturation level if the diaper was not disconnected from the sensor.

2. The method of claim 1, wherein the message from the front-end sensor includes the resistance of the diaper.

3. The method of claim 1, wherein the message from the front-end sensor includes:
the percent change from the previous resistance of the diaper; and
the percent change from the previous capacitance of the diaper.

4. The method of claim 1, wherein the message from the front-end sensor includes the capacitance of the diaper.

5. The method of claim 1, wherein the message from the front-end sensor includes:
the sensor pull up resistance used; and
the sensor sample count used to determine the capacitance.

6. The method of claim 1, wherein processing the message includes determining if any prior messages have been missed.

7. The method of claim 1, wherein the flag in the message can include a diaper change flag.

8. The method of claim 1, wherein the flag in the message can include a saturation change flag.

9. The method of claim 1, wherein estimating the saturation level includes looking up the saturation level in a look up table based on the information in the received message.

10. The method of claim 8, wherein the lookup table used depends on the salinity of the diaper.

11. The method of claim 8, wherein the lookup table used depends on the compression of the diaper.

12. The method of claim 8, wherein once the lookup table is selected it is used for the life of the diaper.

13. The method of claim 1, wherein estimating the saturation level includes keeping a previous saturation level if a current saturation level is less than the previous saturation level by less than a threshold amount.

14. A method for determining a diaper saturation level, the method comprising:
receiving a message from a front-end sensor;
processing the messages;
checking the messages for flags;
determining if the diaper has been disconnected from the sensor;
resetting the saturation level to zero if the diaper has been disconnected;
estimating the saturation level if the diaper was not disconnected from the sensor;
determining whether a user should be notified about the saturation level of the diaper; and
notifying a user of the saturation level if the determination of whether a user should be notified is positive.

15. The method of claim 14, wherein determining whether a user should be notified about the saturation level includes determining if the saturation level increased beyond a threshold amount.

16. The method of claim 14, wherein determining whether a user should be notified about the saturation level includes determining if the saturation level decreased beyond a threshold amount.

17. A system for determining a saturation level in a diaper, the system comprising:
a front-end sensor configured to be placed within a diaper, wherein the front-end sensor is configured to allow a simultaneous estimation of:
the resistance within the diaper; and
the capacitance within the diaper; and
a software application on an external device, wherein the software application:
receives a message from a front-end sensor;
processes the messages;
checks the messages for flags;
determines if the diaper has been disconnected from the sensor;
resets the saturation level to zero if the diaper has been disconnected;
estimates the saturation level if the diaper was not disconnected from the sensor;
determines whether a user should be notified about the saturation level of the diaper; and
notifies a user of the saturation level if the determination of whether a user should be notified is positive.

18. The system of claim 16, wherein the external device is a user's phone.

19. The system of claim 16, wherein the external device is a website.

20. The system of claim 16, wherein the external device can show a usage history.
